(19) United States
(12) Patent Application Publication
(10) Pub. No.: US 2014/0024115 A1
(43) Pub. Date: Jan. 23, 2014

(54) METHODS OF COMBINING MESENCHYMAL STEM CELLS AND CARTILAGE CONTAINING ALLOGRAFTS, AND PRODUCTS OF COMBINED MESENCHYMAL STEM CELLS AND CARTILAGE CONTAINING ALLOGRAFTS

(71) Applicants: Simon Bogdansky, Littleton, CO (US); Yaling Shi, Larkspur, CO (US); Brent Atkinson, Littleton, CO (US)

(72) Inventors: Simon Bogdansky, Littleton, CO (US); Yaling Shi, Larkspur, CO (US); Brent Atkinson, Littleton, CO (US)

(73) Assignee: ALLOSOURCE, Centennial, CO (US)

(21) Appl. No.: 12/965,335

(22) Filed: Dec. 10, 2010

Related U.S. Application Data
(63) Continuation-in-part of application No. 12/612,583, filed on Nov. 4, 2009.
(60) Provisional application No. 61/285,463, filed on Dec. 10, 2009.

(51) Int. Cl. C12N 5/0775

(52) U.S. Cl. 435/381; 435/378; 435/395

(57) ABSTRACT

There is disclosed a method of combining mesenchymal stem cells (MSCs) with an osteochondral allograft. In an embodiment, the method includes obtaining adipose tissue having MSCs with unwanted cells. The tissue is digested to form a cell suspension having MSCs and unwanted cells. The suspension is added to seed the allograft. The MSCs are allowed to attach to the allograft. There is disclosed an allograft product including MSCs with an osteochondral allograft. There is disclosed a method of combining MSCs with decellularized, morselized cartilage. In an embodiment, the method includes obtaining adipose tissue having MSCs with unwanted cells. The tissue is digested to form a cell suspension having MSCs and unwanted cells. The suspension is added to seed the cartilage. The MSCs are allowed to attach to the cartilage to. There is disclosed an allograft product including MSCs with morselized cartilage. Other embodiments are also disclosed.

### REFERENCE TO PENDING PRIOR PATENT APPLICATION

This application claims the benefit under 35 U.S.C. 119 (e) of U.S. Provisional Patent Application No. 61/285,463, filed Dec. 10, 2009 by Simon Bogdansky for “METHOD FOR IMPROVING OSTEOCHONDRAL ALLGRAFTS EMPLOYING ADIPOSE TISSUE-DERIVED STROMAL CELLS”. This patent application is a continuation-in-part of pending prior U.S. patent application Ser. No. 12/612,583 filed Nov. 4, 2009 by Yaling Shi for ALLOGRAFTS COMBINED WITH TISSUE DERIVED STEM CELLS FOR BONE HEALING. The above-identified patent applications are hereby incorporated herein by reference.

### BACKGROUND

Regenerative medicine requires an abundant source of human adult stem cells that can be readily available at the point of care.

Adipose-derived stem cells (ASCs), which can be obtained in large quantities, have been utilized as cellular therapy for the induction of bone formation in tissue engineering strategies.

Allografts may be combined with stem cells. This requires a significant amount of tissue processing and cellular processing prior to seeding the allograft substrate.

Allografts seeded with living cells generally provide better surgical results.

### SUMMARY OF THE INVENTION

In an embodiment, there is provided a method of combining mesenchymal stem cells with an osteochondral allograft, the method comprising obtaining adipose tissue having the mesenchymal stem cells together with unwanted cells; digesting the adipose tissue to form a cell suspension having the mesenchymal stem cells and the unwanted cells; adding the cell suspension with the mesenchymal stem cells to seed the osteochondral allograft so as to form a seeded osteochondral allograft; and allowing the cell suspension to adhere to the osteochondral allograft for a period of time to allow the mesenchymal stem cells to attach.

In another embodiment, there is provided an allograft product including a combination of mesenchymal stem cells with an osteochondral allograft, and the combination manufactured by obtaining adipose tissue having the mesenchymal stem cells together with unwanted cells; digesting the adipose tissue to form a cell suspension having the mesenchymal stem cells and the unwanted cells; adding the cell suspension with the mesenchymal stem cells to seed the osteochondral allograft so as to form a seeded osteochondral allograft; and allowing the cell suspension to adhere to the seeded osteochondral allograft for a period of time to allow the mesenchymal stem cells to attach.

In still another embodiment, there is provided a method of combining mesenchymal stem cells with an osteochondral allograft, the method comprising obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the osteochondral allograft so as to form a seeded osteochondral allograft; and allowing the cell suspension to adhere to seeded osteochondral allograft for a period of time to allow the mesenchymal stem cells to attach.

In yet another embodiment, there is provided an allograft product including a combination of mesenchymal stem cells with an osteochondral allograft, and the combination manufactured by obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the osteochondral allograft so as to form a seeded osteochondral allograft; and allowing the cell suspension to adhere to the osteochondral allograft for a period of time to allow the mesenchymal stem cells to attach.

In an embodiment, there is provided a method of combining mesenchymal stem cells with decellularized, morselized cartilage, the method comprising obtaining adipose tissue having the mesenchymal stem cells together with unwanted cells; digesting the adipose tissue to form a cell suspension having the mesenchymal stem cells and the unwanted cells; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In another embodiment, there is provided an allograft product including a combination of mesenchymal stem cells with decellularized, morselized cartilage, and the combination manufactured by obtaining adipose tissue having the mesenchymal stem cells together with unwanted cells; digesting the adipose tissue to form a cell suspension having the mesenchymal stem cells and the unwanted cells; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In still another embodiment, there is provided a method of combining mesenchymal stem cells with decellularized, morselized cartilage, the method comprising obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In yet another embodiment, there is provided an allograft product including a combination of mesenchymal stem cells with decellularized, morselized cartilage, and the combination manufactured by obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In an embodiment, there is provided a method of combining mesenchymal stem cells with an osteochondral allograft, the method comprising obtaining the mesenchymal stem cells from adipose tissue of a cadaveric donor; obtaining the osteochondral allograft from the same cadaveric donor; adding the mesenchymal stem cells to seed the osteochondral allograft so as to form a seeded osteochondral allograft; and allowing the cell suspension to adhere to the osteochondral allograft for a period of time to allow the mesenchymal stem cells to attach.

In another embodiment, there is provided an allograft product including a combination of mesenchymal stem cells with an osteochondral allograft, and the combination manufactured by combining mesenchymal stem cells with an osteochondral allograft, the method comprising obtaining the mesenchymal stem cells from adipose tissue of a cadaveric donor; obtaining the osteochondral allograft from the same cadaveric donor; adding the mesenchymal stem cells to seed the osteochondral allograft so as to form a seeded osteochondral allograft; and allowing the cell suspension to adhere to the seeded osteochondral allograft for a period of time to allow the mesenchymal stem cells to attach.

In an embodiment, there is provided a method of combining mesenchymal stem cells with decellularized, morselized cartilage, the method comprising obtaining the mesenchymal stem cells from adipose tissue of a cadaveric donor; obtaining the morselized cartilage from the same cadaveric donor; adding the mesenchymal stem cells to seed the morselized cartilage so as to form a seeded osteochondral allograft; and allowing the cell suspension to adhere to the decellularized, morselized cartilage for a period of time to allow the mesenchymal stem cells to attach.

In another embodiment, there is provided an allograft product including a combination of mesenchymal stem cells with decellularized, morselized cartilage, and the combination manufactured by obtaining the mesenchymal stem cells from adipose tissue of a cadaveric donor; obtaining the morselized cartilage from the same cadaveric donor; adding the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In one embodiment, there is disclosed a method of combining mesenchymal stem cells with cartilage, the method comprising obtaining the mesenchymal stem cells from adipose tissue of a cadaveric donor; obtaining the cartilage from the same cadaveric donor; adding the mesenchymal stem cells to seed the cartilage so as to form a seeded cartilage; and allowing the cell suspension to adhere to the mesenchymal stem cells and the cartilage for a period of time to allow the mesenchymal stem cells to attach.

In another embodiment, there is disclosed an allograft product including a combination of mesenchymal stem cells with cartilage, and the combination manufactured by obtaining the mesenchymal stem cells from adipose tissue of a cadaveric donor; obtaining the cartilage from the same cadaveric donor; adding the mesenchymal stem cells to seed the cartilage so as to form a seeded cartilage; and allowing the cell suspension to adhere to the mesenchymal stem cells and the cartilage for a period of time to allow the mesenchymal stem cells to attach.

Other embodiments are also disclosed.

### BRIEF DESCRIPTION OF THE DRAWINGS

Illustrative embodiments of the invention are illustrated in the drawings, in which:

FIG. 1 illustrates a flow chart of an exemplary method of combining mesenchymal stem cells with an osteochondral allograft;

FIG. 2 illustrates a flow chart of an exemplary method of combining mesenchymal stem cells with decellularized, morselized cartilage;

FIG. 3 illustrates an exemplary osteochondral allograft;

FIG. 4 illustrates H&E staining of a cartilage control sample; and

FIG. 5 illustrates H&E staining of adiposed-derived stem cells seeded cartilage.

### DETAILED DESCRIPTION

Unless otherwise described, human adult stem cells are generally referred to as mesenchymal stem cells or MSCs. MSCs are pluripotent cells that have the capacity to differentiate in accordance with at least two discrete development pathways. Adipose-derived stem cells or ASCs are stem cells that are derived from adipose tissue. Stromal Vascular Fraction or SVF generally refers to the centrifuged cell pellet obtained after digestion of tissue containing MSCs. In one embodiment, the SVF pellet may include multiple types of stem cells. These stem cells may include, for example, one or more of hematopoietic stem cells, epithelial stem cells, and mesenchymal stem cells. In an embodiment, mesenchymal stem cells are filtered from other stem cells by their adherence to a osteochondral graft (or cartilage or morselized cartilage), while the other stem cells (i.e., unwanted cells) do not adhere to the osteochondral graft (or cartilage or morselized cartilage). Other cells that do not adhere to the osteochondral graft (or cartilage or morselized cartilage) may also be included in these unwanted cells.

Adipose derived stem cells may be isolated from cadavers and characterized using flow cytometry and tri-lineage differentiation (osteogenesis, chondrogenesis and adipogenesis). The final product may be characterized using histology for microstructure and biochemical assays for cell count. This consistent cell-based product may be useful for osteochondral graft (or cartilage or morselized cartilage) regeneration.

Tissue engineering and regenerative medicine approaches offer great promise to regenerate bodily tissues. The most widely studied tissue engineering approaches, which are based on seeding and in vitro culturing of cells within the scaffold before implantation, is the cell source and the ability to control cell proliferation and differentiation. Many researchers have demonstrated that adipose tissue-derived stem cells (ASCs) possess multiple differentiation capacities. See, for example, the following, which are incorporated by reference:
Rada, T., R. L. Reis, and M. E. Gomes, Adipose Tissue-Derived Stem Cells and Their Application in Bone and Cartilage Tissue Engineering. Tissue Eng Part B Rev, 2009.
Ahn, H. H., et al., In vivo osteogenic differentiation of human adipose-derived stem cells in an injectable in situ-forming gel scaffold. Tissue Eng Part A, 2009. 15(7): p. 1821-32.
Anghileri, E., et al., Neuronal differentiation potential of human adipose-derived mesenchymal stem cells. Stem Cells Dev, 2008. 17(5): p. 909-16.
Arnalich-Montiel, F., et al., Adipose-derived stem cells are a source for cell therapy of the corneal stroma. Stem Cells, 2008. 26(2): p. 570-9.
Bunnell, B. A., et al., Adipose-derived stem cells: isolation, expansion and differentiation. Methods, 2008. 45(2): p. 115-20.
Chen, R. B., et al., [Differentiation of rat adipose-derived stem cells into smooth-muscle-like cells in vitro]. Zhonghua Nan Ke Xue, 2009. 15(5): p. 425-30.
Cheng, N. C., et al., Chondrogenic differentiation of adipose-derived adult stem cells by a porous scaffold derived from native articular cartilage extracellular matrix. Tissue Eng Part A, 2009. 15(2): p. 231-41.
Cui, L., et al., Repair of cranial bone defects with adipose derived stem cells and coral scaffold in a canine model. Biomaterials, 2007. 28(36): p. 5477-86.
de Girolamo, L., et al., Osteogenic differentiation of human adipose-derived stem cells: comparison of two different inductive media. J Tissue Eng Regen Med, 2007. 1(2): p. 154-7.
Elabd, C., et al., Human adipose tissue-derived multipotent stem cells differentiate in vitro and in vivo into osteocyte-like cells. Biochem Biophys Res Commun, 2007. 361(2): p. 342-8.
Flynn, L., et al., Adipose tissue engineering with naturally derived scaffolds and adipose-derived stem cells. Biomaterials, 2007. 28(26): p. 3834-42.
Flynn, L. E., et al., Proliferation and differentiation of adipose-derived stem cells on naturally derived scaffolds. Biomaterials, 2008. 29(12): p. 1862-71.
Fraser, J. K., et al., Adipose-derived stem cells. Methods Mol Biol, 2008. 449: p. 59-67.
Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. Curr Top Dev Biol, 2003. 58: p. 137-60.
Jin, X. B., et al., Tissue engineered cartilage from hTGF beta2 transduced human adipose derived stem cells seeded in PLGA/alginate compound in vitro and in vivo. J Biomed Mater Res A, 2008. 86(4): p. 1077-87.
Kakudo, N., et al., Bone tissue engineering using human adipose-derived stem cells and honeycomb collagen scaffold. J Biomed Mater Res A, 2008. 84(1): p. 191-7.
Kim, H. J. and G. I. Im, Chondrogenic differentiation of adipose tissue-derived mesenchymal stem cells: greater doses of growth factor are necessary. J Orthop Res, 2009. 27(5): p. 612-9.
Kingham, P. J., et al., Adipose-derived stem cells differentiate into a Schwann cell phenotype and promote neurite outgrowth in vitro. Exp Neurol, 2007. 207(2): p. 267-74.
Mehlhorn, A. T., et al., Chondrogenesis of adipose-derived adult stem cells in a poly-lactide-co-glycolide scaffold. Tissue Eng Part A, 2009. 15(5): p. 1159-67.
Merceron, C., et al., Adipose-derived mesenchymal stem cells and biomaterials for cartilage tissue engineering. Joint Bone Spine, 2008. 75(6): p. 672-4.
Mischen, B. T., et al., Metabolic and functional characterization of human adipose-derived stem cells in tissue engineering. Plast Reconstr Surg, 2008. 122(3): p. 725-38.
Mizuno, H., Adipose-derived stem cells for tissue repair and regeneration: ten years of research and a literature review. J Nippon Med Sch, 2009. 76(2): p. 56-66.
Tapp, H., et al., Adipose-Derived Stem Cells: Characterization and Current Application in Orthopaedic Tissue Repair. Exp Biol Med (Maywood), 2008.
Tapp, H., et al., Adipose-derived stem cells: characterization and current application in orthopaedic tissue repair. Exp Biol Med (Maywood), 2009. 234(1): p. 1-9.
van Dijk, A., et al., Differentiation of human adipose-derived stem cells towards cardiomyocytes is facilitated by laminin. Cell Tissue Res, 2008. 334(3): p. 457-67.
Wei, Y., et al., A novel injectable scaffold for cartilage tissue engineering using adipose-derived adult stem cells. J Orthop Res, 2008. 26(1): p. 27-33.
Wei, Y., et al., Adipose-derived stem cells and chondrogenesis. Cytotherapy, 2007. 9(8): p. 712-6.
Zhang, Y. S., et al., [Adipose tissue engineering with human adipose-derived stem cells and fibrin glue injectable scaffold]. Zhonghua Yi Xue Za Zhi, 2008. 88(38): p. 2705-9.

Additionally, adipose tissue is probably the most abundant and accessible source of adult stem cells. Adipose tissue derived stem cells have great potential for tissue regeneration. Nevertheless, ASCs and bone marrow-derived stem cells (BMSCs) are remarkably similar with respect to growth and morphology, displaying fibroblastic characteristics, with abundant endoplasmic reticulum and large nucleus relative to the cytoplasmic volume. See, for example, the following, which are incorporated by reference:
Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. Curr Top Dev Biol, 2003. 58: p. 137-60.
Strem, B. M., et al., Multipotential differentiation of adipose tissue-derived stem cells. Keio J Med, 2005. 54(3): p. 132-41.
De Ugarte, D. A., et al., Comparison of multi-lineage cells from human adipose tissue and bone marrow. Cells Tissues Organs, 2003. 174(3): p. 101-9.
Hayashi, O., et al., Comparison of osteogenic ability of rat mesenchymal stem cells from bone marrow, periosteum, and adipose tissue. Calcif Tissue Int, 2008. 82(3): p. 238-47.
Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
Lin, L., et al., Comparison of osteogenic potentials of BMP4 transduced stem cells from autologous bone marrow and fat tissue in a rabbit model of calvarial defects. Calcif Tissue Int, 2009. 85(1): p. 55-65.
Niemeyer, P., et al., Comparison of immunological properties of bone marrow stromal cells and adipose tissue-derived stem cells before and after osteogenic differentiation in vitro. Tissue Eng, 2007. 13(1): p. 111-21.
Noel, D., et al., Cell specific differences between human adipose-derived and mesenchymal-stromal cells despite similar differentiation potentials. Exp Cell Res, 2008. 314(7): p. 1575-84.
Yoo, K. H., et al., Comparison of immunomodulatory properties of mesenchymal stem cells derived from adult human tissues. Cell Immunol, 2009.
Yoshimura, H., et al., Comparison of rat mesenchymal stem cells derived from bone marrow, synovium, periosteum, adipose tissue, and muscle. Cell Tissue Res, 2007. 327(3): p. 449-62.

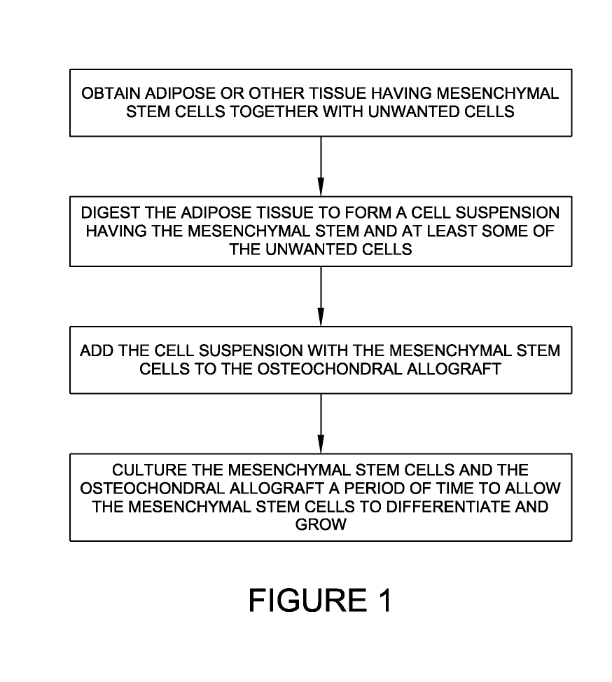

FIG. 1 is a flow chart of a process for combining an osteochondral allograft with stem cells. In an embodiment, a stromal vascular fraction may be used to seed the allograft. It should be apparent from the present disclosure that the term “seed” relates to addition and placement of the stem cells within, or at least in attachment to, the allograft, but is not limited to a specific process.

In an exemplary embodiment, a method of combining mesenchymal stem cells with an osteochondral allograft is provided. The method may include obtaining adipose tissue having the mesenchymal stem cells together with unwanted cells. Unwanted cells may include hematopoietic stem sells and other stromal cells. The method may further include digesting the adipose tissue to form a cell suspension having the mesenchymal stem cells and at least some or all of the unwanted cells. In another embodiment, this may be followed by negatively depleting some of the unwanted cells and other constituents to concentrate mesenchymal stem cells.

Next, the method includes adding the cell suspension with the mesenchymal stem cells to seed the osteochondral allograft. This may be followed by allowing the cell suspension to adhere to the osteochondral allograft for a period of time to allow the mesenchymal stem cells to attach. In order to provide a desired product, the method may include rinsing the seeded osteochondral allograft to remove the unwanted cells from the seeded ostechondral allograft.

In one embodiment, an allograft product may include a combination of mesenchymal stem cells with an osteochondral allograft such that the combination is manufactured by the above exemplary embodiment.

In an embodiment, the adipose tissue may be obtained from a cadaveric donor. A typical donor yields 2 liters of adipose containing 18 million MSCs. In one embodiment, an osteochondral allograft may be from the same cadaveric donor as the adipose tissue. In another embodiment, the adipose tissue may be obtained from a patient that will be undergoing the cartilage or osteochondral replacement/regeneration surgery. In addition, both the osteochondral graft (or cartilage or morselized cartilage) and the adipose tissue may be obtained from the same cadaveric donor. Adipose cells may be removed using liposuction. Other sources, and combination of sources, of adipose tissue, other tissues, and osteochondral allografts may be utilized.

Optionally, the adipose tissue may be washed prior to or during digestion. Washing may include using a thermal shaker at 75 RPM at 37° C. for at least 10 minutes. Washing the adipose tissue may include washing with a volume of PBS substantially equal to the adipose tissue. In an embodiment, washing the adipose tissue includes washing with the PBS with 1% penicillin and streptomycin at about 37° C.

For example, washing the adipose tissue may include agitating the tissue and allowing phase separation for about 3 to 5 minutes. This may be followed by aspirating off a supernatant solution. The washing may include repeating washing the adipose tissue multiple times until a clear infranatant solution is obtained. In one embodiment, washing the adipose tissue may include washing with a volume of growth media substantially equal to the adipose tissue.

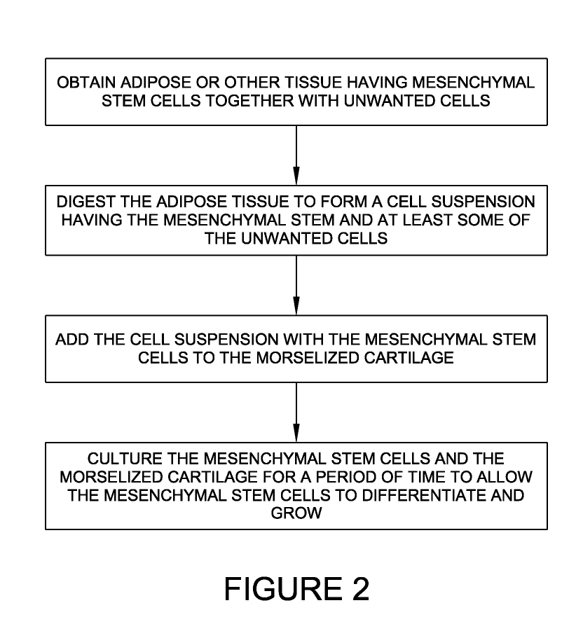

FIG. 2 is a flow chart of a process for combining morselized cartilage with stem cells. In an embodiment, a stromal vascular fraction may be used to seed the allograft.

In another exemplary embodiment, a method of combining mesenchymal stem cells with decellularized, morselized cartilage is provided. The method may include obtaining adipose tissue having the mesenchymal stem cells together with unwanted cells. Unwanted cells may include hematopoietic stem sells and other stromal cells. The method may further include digesting the adipose-derived tissue to form a cell suspension having the mesenchymal stem cells and the unwanted cells. In another embodiment, this may be followed by naturally selecting MSCs and depleting some of the unwanted cells and other constituents to concentrate mesenchymal stem cells.

Next, the method includes adding the cell suspension with the mesenchymal stem cells to the morselized cartilage. This may be followed by allowing the cell suspension to adhere to the mesenchymal stem cells and the morselized cartilage for a period of time to allow the mesenchymal stem cells to attach In order to provide a desired product, the method may include rinsing the seeded morselized cartilage to remove the unwanted cells from the seeded morselized cartilage.

In one embodiment, an allograft product may include a combination of mesenchymal stem cells with decellularized, morselized cartilage such that the combination is manufactured by the above exemplary embodiment.

In an embodiment, the adipose tissue may be obtained from a cadaveric donor. A typical donor yields 2 liters of adipose containing 18 million MSCs. In one embodiment, morselized cartilage may be from the same cadaveric donor as the adipose tissue. In another embodiment, the adipose tissue may be obtained from a patient. In addition, both the osteochondral graft (or cartilage or morselized cartilage) and the adipose tissue may be obtained from the same cadaveric donor. Adipose cells may be removed using liposuction. Other sources, and combination of sources, of adipose tissue, other tissues, and morselized cartilage may be utilized.

Optionally, the adipose tissue may be washed prior to or during digestion. Washing may include using a thermal shaker at 75 RPM at 37° C. for at least 10 minutes. Washing the adipose tissue may include washing with a volume of PBS substantially equal to the adipose tissue. In an embodiment, washing the adipose tissue includes washing with the PBS with 1% penicillin and streptomycin at about 37° C.

For example, washing the adipose tissue may include agitating the tissue and allowing phase separation for about 3 to 5 minutes. This may be followed by aspirating off a supernatant solution. The washing may include repeating washing the adipose tissue multiple times until a clear infranatant solution is obtained. In one embodiment, washing the adipose tissue may include washing with a volume of growth media substantially equal to the adipose tissue.

Digesting the cell suspension may include making a collagenase I solution, and filtering the solution through a 0.2 μm filter unit, mixing the adipose tissue with the collagenase I solution, and adding the cell suspension mixed with the collagenase I solution to a shaker flask. Digesting the cell suspension may further include placing the shaker with continuous agitation at about 75 RPM for about 45 to 60 minutes so as to provide the adipose tissue with a visually smooth appearance.

Digesting the cell suspension may further include aspirating supernatant containing mature adipocytes so as to provide a pellet, which may be referred to as a stromal vascular fraction. (See, for example, FIG. 2.) Prior to seeding, a lab sponge or other mechanism may be used to pat dry cells from the pellet.

In various embodiments, adding the cell suspension with the mesenchymal stem cells to the osteochondral allograft or the morselized cartilage may include using a cell pellet for seeding onto the osteochondral graft (or cartilage or morselized cartilage). In an embodiment, adding the cell suspension with the mesenchymal stem cells to the osteochondral allograft or the morselized cartilage may include using a cell pellet for seeding onto the osteochondral graft (or cartilage or morselized cartilage). In another embodiment, adding the cell suspension with the mesenchymal stem cells to the osteochondral allograft or the morselized cartilage may include using a cell pellet for seeding onto the osteochondral allograft or the morselized cartilage. In another embodiment, adding the cell suspension with the mesenchymal stem cells to seed the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage.

In various embodiments, the method may include placing the osteochondral graft (or cartilage or morselized cartilage) into a cryopreservation media after rinsing the osteochondral allograft or the morselized cartilage. This cryopreservation media may be provided to store the final products. For example, the method may include maintaining the osteochondral allograft or the morselized cartilage into a frozen state after rinsing the osteochondral allograft or the morselized cartilage to store the final products. The frozen state may be at about negative 80° C.

In another embodiment, Ficoll density solution may be utilized. For example, negatively depleting the concentration of the mesenchymal stem cells may include adding a volume of PBS and a volume of Ficoll density solution to the adipose solution. The volume of PBS may be 5 ml and the volume of Ficoll density solution may be 25 ml with a density of 1.073 g/ml. Negatively depleting the concentration of the mesenchymal stem cells may also include centrifuging the adipose solution at about 1160 g for about 30 minutes at about room temperature. In one embodiment, the method may include stopping the centrifuging the adipose solution without using a brake.

Negatively depleting the concentration of the mesenchymal stem cells is optional and may next include collecting an upper layer and an interface containing nucleated cells, and discarding a lower layer of red cells and cell debris. Negatively depleting the concentration of the mesenchymal stem cells may also include adding a volume of D-PBS of about twice an amount of the upper layer of nucleated cells, and inverting a container containing the cells to wash the collected cells. Negatively depleting the concentration of the mesenchymal stem cells may include centrifuging the collected cells to pellet the collected cells using the break during deceleration.

In an embodiment, negatively depleting the concentration of the mesenchymal stem cells may further include centrifuging the collected cells at about 900 g for about 5 minutes at about room temperature. Negatively depleting some of the unwanted cells may include discarding a supernatant after centrifuging the collected cells, and resuspending the collected cells in a growth medium.

In various embodiments, adding the cell suspension with the mesenchymal stem cells to the osteochondral allograft or the morselized cartilage may include adding the cell pellet onto the osteochondral allograft or the morselized cartilage. Adding the solution with the mesenchymal stem cells to the osteochondral allograft or the morselized cartilage may include adding cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In an embodiment, adding the cell suspension with the mesenchymal stem cells to the osteochondral allograft or the morselized cartilage includes adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage.

In various embodiments, the method may further include placing the osteochondral allograft or the morselized cartilage into a cryopreservation media after rinsing the osteochondral allograft or the morselized cartilage. This cryopreservation media may be provided to store the final products. The method may include maintaining the osteochondral allograft or the morselized cartilage into a frozen state after rinsing the osteochondral allograft or the morselized cartilage to store the final products. The frozen state may be at about negative 80° C.

The cell suspension is allowed to adhere to seeded allografts for a period of time to allow the mesenchymal stem cells to attach to the osteochondral allograft or the morselized cartilage. The unwanted cells may be rinsed and removed from the osteochondral allograft or the morselized cartilage. After culturing, a lab sponge or other mechanism may be used to pat dry the cells.

Previous methods used autogenous osteochondral grafts, wherein a graft from one area of a donor knee was transplanted to same donor knee, but to an area that was damaged. However, this method causes trauma to the patient and creates a new area that is damaged. Allografts are currently used that prevent the trauma caused by autografts. Non-processed osteochondral allografts suffer from being immune reactive. Processed osteochondral allografts suffer from either having no viable cells, reduced viability, or fully differentiated cells that are not capable of undergoing regeneration. Thus, there is a need to provide a cartilage graft that contains viable MSCs to recapitulate the regenerative cascade.

The surface of cartilage, by its very nature, is not adherent to cells. The mesenchymal stem cells are anchorage dependent, but this has been defined as being adherent to tissue culture plastic, not a biological tissue like cartilage. Surprisingly, the methods provided herein permit viable MSCs that bind to cartilage.

The methods provided herein describe the allograft processing that allows MSCs to adhere to the scaffold. The method in the example demonstrates a blending and processing method that removes cells from the cartilage graft such that viable MSCs can adhere.

The mesenchymal stem cells are non-immunogenic and regenerate cartilage of the osteochondral allograft or the morselized cartilage. The unwanted cells are generally anchorage independent. This means that the unwanted cells generally do not adhere to the osteochondral allograft or the morselized cartilage. The unwanted cells may be immunogenic. For cell purification during a rinse, mesenchymal stem cells adhere to the osteochondral allograft or the morselized cartilage while unwanted cells, such as hematopoietic stem sells, are rinsed away leaving a substantially uniform population of mesenchymal stem cells on the osteochondral graft (or cartilage or morselized cartilage).

The ability to mineralize the extracellular matrix and to generate cartilage is not unique to MSCs. In fact, ASCs possess a similar ability to differentiate into chondrocytes under similar conditions. Human ASCs offer a unique advantage in contrast to other cell sources. The multipotent characteristics of ASCs, as wells as their abundance in the human body, make these cells a desirable source in tissue engineering applications.

In addition, this method and combination product involve processing that does not alter the relevant biological characteristics of the tissue. Processing of the adipose/stem cells may involve the use of antibiotics, cell media, collagenase. None of these affects the relevant biological characteristics of the stem cells. The relevant biological characteristics of these mesenchymal stem cells are centered on renewal and repair. The processing of the stem cells does not alter the cell's ability to continue to differentiate and repair.

In the absence of stimulation or environmental cues, mesenchymal stem cells (MSCs) remain undifferentiated and maintain their potential to form tissue such as bone, cartilage, fat, and muscle. Upon attachment to an osteoconductive matrix, MSCs have been shown to differentiate along the osteoblastic lineage in vivo. See, for example, the following, which are incorporated by reference:
Arinzeh T L, Peter S J, Archambault M P, van den Bos C, Gordon S, Kraus K, Smith A, Kadiyala S. Allogeneic mesenchymal stem cells regenerate bone in a critical sized canine segmental defect. J Bone Joint Surg Am. 2003; 85-A:1927-35.
Bruder S P, Kurth A A, Shea M, Hayes W C, Jaiswal N, Kadiyala S. Bone regeneration by implantation of purified, culture-expanded human mesenchymal stem cells, J Orthop Res. 1998; 16:155-62.

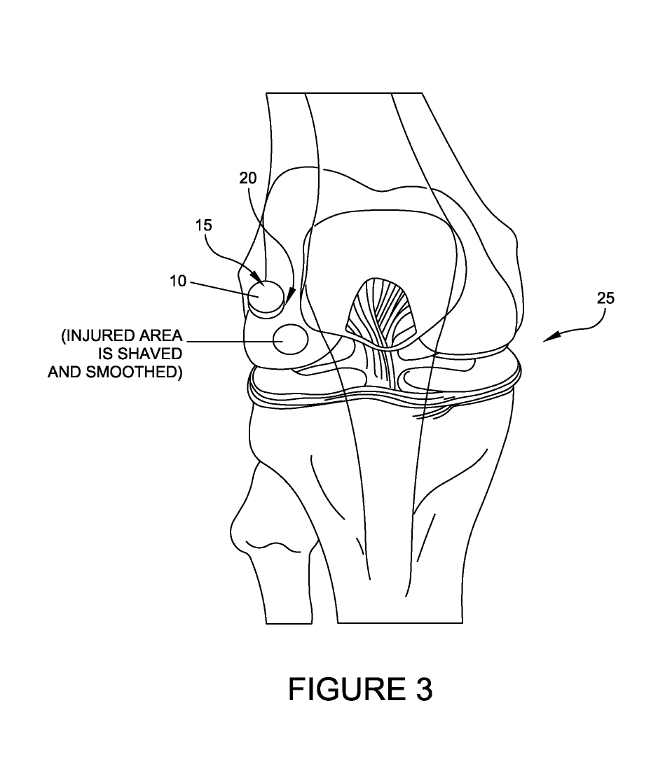

Referring to FIG. 3, and in an embodiment, there is illustrated an osteochondral allograft 10, which may include cartilage 15 and bone 20 from a cadaver. Osteochondral allograft may be placed in the area of a knee 25 or other joint where cartilage is missing. This technique may be used where there is a large area of cartilage that is missing or if there both bone and cartilage are missing. The donor allograft must be tested for contamination, which may include bacteria, hepatitis, and HIV. Having a single donor for both the osteochondral allograft and adipose-derived mesenchymal stem cells may reduce testing burdens and minimize other potential issues.

### Example

Cartilage Combined with Adipose-Derived Stem Cells

The objective was to determine whether adipose derived stem cells adhere to processed and ground articular cartilage.

ASCs adhere to cartilage, and promote cartilage repair and regeneration.

Experiment Design:

| Cartilage with | Cartilage w/o | | |
|---|---|---|---|
| ASCs | ASCs | ASCs only | Medium only |
| n = 3, 36 h | n = 3, 36 h | n = 3, 36 h | n = 3, 36 h |
| incubation | incubation | incubation | incubation |

Materials and Methods:

Sample Preparation: Cartilage pieces previously shaved from knee articulating surface and frozen at −80° C. were thawed and blended (Waring Blender) for approximately 2 minutes on “Hi” (22,000 rpms) while submerged in PBS. Resulting particles were approximately 1 mm×2-3 mm×1 mm. The particles were then rinsed and drained in a sieve and were separated into six 5 ml samples and placed into a 6-well plate. Prior to seeding, cartilage samples were patted dry with sterile gauze. Three wells containing cartilage were each seeded with 200 μl cell suspension. The other three wells containing cartilage only were left as unseeded controls. An empty 6-well plate was seeded in the same fashion with three wells receiving cells and three wells without cells. The wells were incubated for an hour at 37° C. and 5% CO2 in a humidified incubator, then submerged in 5 ml DMEM-F12/10% FBS/1% PSA and incubated for 36 hrs. All the samples in the 6-well plates were tested using CCK-8 assay for cell counts and the cartilage samples were collected for histology.

Cell Count: CCK-8 Assay

Cell Counting Kit-8 (CCK-8, Dojindo Molecular Technologies, Maryland) allows sensitive colorimetric assays for the determination of the number of viable cells in cell proliferation assays. The amount of the formazan dye generated by the activity of dehydrogenases in cells is directly proportional to the number of living cells. The samples were rinsed with PBS and then patted dry. Growth medium and CCK-8 solution were added into wells at a ratio of 10:1 cultured at 37° C. for 2 hours and evaluated in a plate reader with excitation set to 460 nm and emission set to 650 nm. The results were interpolated from a standard curve based on ASCs only (passage=1).

Histology

The cartilage samples were fixed in 10% neutral buffered formalin (Sigma, St. Louis, Mo.) for 48 h, put in a processor (Citadel 2000; Thermo Shandon, Pittsburgh, Pa.) overnight, and embedded in paraffin. Sections were cut to 5 μm and mounted onto glass slides and stained with hematoxylin and eosin (H&E). Conventional light microscopy was used to analyze sections for matrix and cell morphology.

Results

Cell Counts: The number of cells on cartilage was significantly different from ASCs-only controls which were cultured in the 6 well plates.

| | ASCs + Cartilage | Cartilage only | Medium Only |
|---|---|---|---|
| Number of | 4,665 | 0 | 0 |
| Viable Cells | | | |

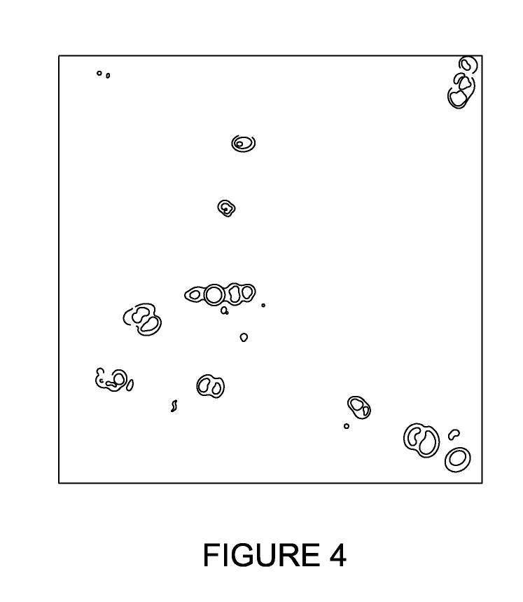

FIG. 4 illustrates H&E staining of cartilage control (10× magnification). Note that there were no live cells in the voids of the ground cartilage matrix.

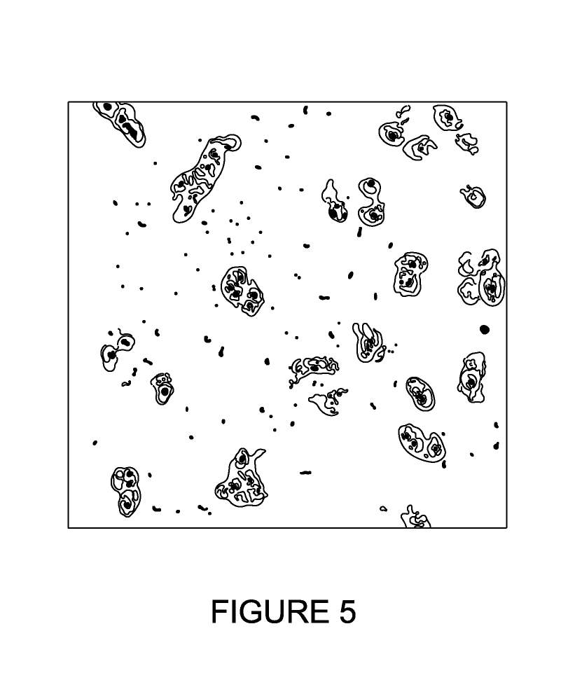

FIG. 5 illustrates H&E staining of ASCs seeded cartilage (10× magnification). Note the live cell nuclei in the voids.

In the cartilage only control, there were no live cells, only the dead cell debris was discovered. The cells seemed to be all dead and left the voids behind. In the ASCs seeded cartilage, it seemed that all the seeded cells repopulated the voids left by pre-existing cells from the cartilage. There were no live cells on the cartilage surface that lacked decellularized zones.

### CONCLUSIONS

ASCs did not adhere to the cartilage matrix, however, they repopulated in the voids left from pre-existing cartilage cells.

1. A method of combining mesenchymal stem cells with an osteochondral allograft, the method comprising:
obtaining adipose tissue having the mesenchymal stem cells together with unwanted cells;
digesting the adipose tissue to form a cell suspension having the mesenchymal stem cells and the unwanted cells;
adding the cell suspension with the mesenchymal stem cells to the osteochondral allograft so as to form a seeded osteochondral allograft; and
allowing the cell suspension to adhere to the osteochondral allograft for a period of time to allow the mesenchymal stem cells to attach.

2. A method in accordance with claim 1, wherein the step of obtaining the adipose tissue includes recovery from a cadaveric donor.

3. A method in accordance with claim 1, wherein the osteochondral allograft is from a cadaveric donor, and the step of obtaining the adipose tissue includes recovery from the same cadaveric donor as the osteochondral allograft.

4. A method in accordance with claim 1, wherein the step of digesting the adipose tissue includes making a collagenase I solution, and filtering the solution through a 0.2 μm filter unit, mixing the adipose with the collagenase I solution, and adding the collagenase I solution to a shaker flask.

5. A method in accordance with claim 4, wherein the step of digesting the adipose further includes placing the shaker with continuous agitation at about 75 RPM for about 45 to 60 minutes so as to provide the adipose tissue with a visually smooth appearance.

6. A method in accordance with claim 1, wherein the step of digesting the adipose further includes aspirating a supernatant containing mature adipocytes so as to provide a pellet.

7. A method in accordance with claim 6, wherein the step of adding the suspension with the mesenchymal stem cells to seed the osteochondral allograft includes adding the cell suspension onto the cartilage.

8. A method in accordance with claim 7, wherein the step of adding the suspension with the mesenchymal stem cells to seed the osteochondral allograft includes adding the cell suspension into decellularized voids in the osteochondral allograft.

9. A method in accordance with claim 7, wherein the step of adding the suspension with the mesenchymal stem cells to seed the osteochondral allograft includes injecting the suspension into the cartilage.

10. A method in accordance with claim 1, further comprising removing the unwanted cells from the seeded osteochondral allograft.

11. An allograft product including a combination of mesenchymal stem cells with an osteochondral allograft, and the combination manufactured by the method of claim 1.

12. An allograft product in accordance with claim 11, wherein the adipose tissue is recovered from a cadaveric donor, and the osteochondral allograft is recovered from the same cadaveric donor as the adipose tissue.

13. A method of combining mesenchymal stem cells with an osteochondral allograft, the method comprising:
obtaining adipose tissue having the mesenchymal stem cells together with unwanted cells;
digesting the adipose tissue to form a cell suspension having the mesenchymal stem cells and the unwanted cells to acquire a stromal vascular fraction, and the digesting includes:
making a collagenase I solution, and filtering the solution through a 0.2 μm filter unit, mixing the adipose solution with the collagenase I solution, and adding the adipose solution mixed with the collagenase solution to a shaker flask;
placing the shaker with continuous agitation at about 75 RPM for about 45 to 60 minutes so as to provide the adipose tissue with a visually smooth appearance;
aspirating a supernatant containing mature adipocytes so as to provide a pellet;
adding the cell suspension with the mesenchymal stem cells to seed the osteochondral allograft so as to form a seeded osteochondral allograft; and
allowing the cell suspension to adhere to the osteochondral allograft for a period of time to allow the mesenchymal stem cells to attach.

14. An allograft product including a combination of mesenchymal stem cells with an osteochondral allograft, and the combination manufactured by the method of claim 13.

15. An allograft product in accordance with claim 14, wherein the adipose tissue is recovered from a cadaveric donor, and the osteochondral allograft is recovered from the same cadaveric donor as the adipose tissue.

16. A method of combining mesenchymal stem cells with decellularized, morselized cartilage, the method comprising:
obtaining adipose tissue having the mesenchymal stem cells together with unwanted cells;
digesting the adipose tissue to form a stromal vascular fraction comprising a heterogeneous population of the mesenchymal stem cells and the unwanted cells;
adding the stromal vascular fraction comprising the heterogeneous population of the mesenchymal stem cells and the unwanted cells to seed the decellularized, morselized cartilage so as to form seeded decellularized, morselized cartilage;
incubating the seeded decellularized, morselized cartilage for a period of time to allow the mesenchymal stem cells to attach to the decellularized, morselized cartilage; and
rinsing the seeded decellularized, morselized cartilage to remove the unwanted cells from the decellularized morselized cartilage.

17. A method in accordance with claim 16, wherein the step of obtaining the adipose tissue includes recovery from a cadaveric donor.

18. A method in accordance with claim 16, wherein the decellularized, morselized cartilage is from a cadaveric donor, and the step of obtaining the adipose tissue includes recovery from the same cadaveric donor as the decellularized, morselized cartilage.

19. A method in accordance with claim 16, wherein the step of digesting the adipose tissue includes making a collagenase I solution, and filtering the solution through a 0.2 μm filter unit, mixing the adipose with the collagenase I solution, and adding the adipose with the collagenase I solution to a shaker flask.

20. A method in accordance with claim 19, wherein the step of digesting the adipose further includes placing the shaker with continuous agitation at about 75 RPM for about 45 to 60 minutes so as to provide the adipose tissue with a visually smooth appearance.

21. A method in accordance with claim 16, wherein the step of digesting the adipose to form a stromal vascular fraction further includes aspirating a supernatant containing mature adipocytes so as to provide a pellet that is the stromal vascular fraction.

22. A method in accordance with claim 16, wherein the step of adding the stromal vascular fraction comprising the heterogeneous population of the mesenchymal stem cells and the unwanted cells to seed the decellularized, morselized cartilage includes adding the stromal vascular fraction onto pieces of the decellularized, morselized cartilage.

23. A method in accordance with claim 16, wherein the step of adding the stromal vascular fraction comprising the heterogeneous population of the mesenchymal stem cells and the unwanted cells to seed the decellularized, morselized cartilage includes adding the stromal vascular fraction into voids in the pieces of the decellularized, morselized cartilage.

24-25. (canceled)

26. An allograft product including a combination of mesenchymal stem cells with decellularized, morselized cartilage, and the combination manufactured by the method of claim 16.

27. An allograft product in accordance with claim 26, wherein the adipose tissue is recovered from a cadaveric donor, and the decellularized, morselized cartilage is recovered from the same cadaveric donor as the adipose tissue.

28. A method of combining mesenchymal stem cells with decellularized, morselized cartilage, the method comprising:
obtaining adipose tissue having the mesenchymal stem cells together with unwanted cells;
digesting the adipose tissue to form a cell suspension having the mesenchymal stem cells and the unwanted cells to acquire a stromal vascular fraction, and the digesting includes:
making a collagenase I solution, and filtering the solution through a 0.2 μm filter unit, mixing the adipose solution with the collagenase I solution, and adding the adipose solution mixed with the collagenase I solution to a shaker flask;
placing the shaker with continuous agitation at about 75 RPM for about 45 to 60 minutes so as to provide the adipose tissue with a visually smooth appearance;
aspirating a supernatant containing mature adipocytes so as to provide a pellet;
adding the cell suspension with the mesenchymal stem cells to seed the morselized cartilage so as to form seeded morselized cartilage; and
allowing the cell suspension to adhere to the decellularized, morselized cartilage for a period of time to allow the mesenchymal stem cells to attach.

29. An allograft product including a combination of mesenchymal stem cells with decellularized, morselized cartilage, and the combination manufactured by the method of claim 28.

30. An allograft product in accordance with claim 29, wherein the adipose tissue is recovered from a cadaveric donor, and the morselized cartilage is recovered from the same cadaveric donor as the adipose tissue.

31. A method of combining mesenchymal stem cells with an osteochondral allograft, the method comprising:
obtaining the mesenchymal stem cells from adipose tissue of a cadaveric donor;
obtaining the osteochondral allograft from the same cadaveric donor;
adding the mesenchymal stem cells to seed the osteochondral allograft so as to form a seeded osteochondral allograft; and
allowing the cell suspension to adhere to the osteochondral allograft for a period of time to allow the mesenchymal stem cells to attach.

32. An allograft product including a combination of mesenchymal stem cells with an osteochondral allograft, and the combination manufactured by the method of claim 31.

33. A method of combining mesenchymal stem cells with morselized cartilage, the method comprising:
obtaining the mesenchymal stem cells from adipose tissue of a cadaveric donor;
obtaining the morselized cartilage from the same cadaveric donor;
adding the mesenchymal stem cells to seed the morselized cartilage so as to form a seeded morselized cartilage; and
allowing the cell suspension to adhere to the mesenchymal stem cells and the morselized cartilage for a period of time to allow the mesenchymal stem cells to attach.

34. An allograft product including a combination of mesenchymal stem cells with morselized cartilage, and the combination manufactured by the method of claim 32.

35. A method of combining mesenchymal stem cells with cartilage, the method comprising:
obtaining the mesenchymal stem cells from adipose tissue of a cadaveric donor;
obtaining the cartilage from the same cadaveric donor;
adding the mesenchymal stem cells to seed the cartilage so as to form a seeded cartilage; and
allowing the cell suspension to adhere to the mesenchymal stem cells and the cartilage for a period of time to allow the mesenchymal stem cells to attach.

36. An allograft product including a combination of mesenchymal stem cells with cartilage, and the combination manufactured by the method of claim 35.

37. A method in accordance with claim 16, wherein the unwanted cells comprise cells that do not adhere to the decellularized, morselized cartilage.

38. A method in accordance with claim 16, further comprising maintaining the decellularized, morselized cartilage in a frozen state after rinsing the decellularized, morselized cartilage.

39. A method in accordance with claim 38, wherein the frozen state is at about −8° C.
